(19) United States
(12) Patent Application Publication
(10) Pub. No.: US 2007/0065793 A1
(43) Pub. Date: Mar. 22, 2007

(54) Hybrid intelligence in medicine

(76) Inventors: Anuthep Benja-Athon, New York, NY (US); Theda Benja-Athon, Port Washington, NY (US)

Correspondence Address: ANUTHEP BENJA-ATHON, 210 E 36TH ST., NEW YORK, NY, 10016, US

(21) Appl. No.: 11/599,847

(22) Filed: Nov. 16, 2006

Related U.S. Application Data
(63) Continuation-in-part of application No. 11/523,945, filed on Sep. 21, 2006.
(63) Continuation-in-part of application No. 09/191,795, filed on Nov. 13, 1998, now Pat. No. 6,132,218.

(51) Int. Cl. G09B 23/28; G06Q 10/00; A61B 5/00

(52) U.S. Cl. 434/267; 434/272; 600/300; 705/2

(57) ABSTRACT

A method of linking anatomical sites on human images, symptoms and signs relating to the anatomical sites relating to or affected by the symptoms and signs and components of personal, health and health-care data of individuals to diagnoses of diseases and illnesses relating to the sites, symptoms and signs and components of the data wherein anatomical sites, symptoms and signs are selected or expressed by individuals for generating and displaying lists of diagnoses.

This application is a continuation-in-part of U.S. Pat. No. 6,132,218 entitled the Images for Communication of Medical Information in Computer granted Oct. 17, 2000 and a continuation-in-part of Ser. No. 11/523,945 entitled the Networking of Digital Health Information filed Sep. 21, 2006 which is pending.

### FIELD OF INVENTION

A hybridization of computer databases and human thought processes in the generations of diagnoses of diseases and illnesses.

### BACKGROUND OF THE INVENTION

For decades, prior art attempted—albeit failed—to use powerful and super computers to perform and achieve accurate diagnoses (plural of diagnosis) of patients' diseases and illnesses. One of the reasons for the failure of computers to perform and achieve said task is because computers—no matter how powerful—still lack the human faculty of thinking and reasoning.

The present invention is a system and a method of engaging human thought processes with intelligent computer databases comprise personal, health, health-care and medicine data and information to produce the system superior to and innovative than the artificial intelligence sought by prior art.

Specifically, the present invention is a system and a method of engaging human thought processes with intelligent computer databases of health, health-care and medicine data and information to generate and display lists of diagnoses.

More specifically, the present invention provides the uses of a network of computers and servers or networks of computers and servers, the teachings of U.S. Pat. No. 6,132,218 entitled the Images for Communication of Medical Information in Computer granted on Oct. 17, 2000 to the present applicant and of pending patent application Ser. No. 11/523,945 entitled the Networking of digital health information filed Sep. 21, 2006 by the present applicants and the method infra to achieve the goals and objectives of the present invention.

Consequently, first, individuals such as, but not limited to, patients become better-informed and wiser health and health-care buyers and consumers and better patients. Second, they can expeditiously and efficiently find and access physicians and other health-care providers. The socioeconomic benefits are immense.

True to the present applicants' endeavors in delivering the best health-care system for billions of people, the benefits of the present invention for humanity are hugely immense in terms of perpetually improving billions of people's quality of life and standard of living, reducing medical errors and human suffering and saving of millions of lives each year.

### SUMMARY OF THE INVENTION

In artificial intelligence, prior art attempted—albeit failed—to produce accurate diagnoses of patients' diseases and illnesses by powerful and super computers.

The present invention is a system and a method of engaging human thought processes with intelligent computer databases comprise personal, health, health-care and medicine data and information to generate and display lists of diagnoses.

The present invention provides a method of linking anatomical sites display on human images, figurine, figurines and the like, symptoms and signs relating to said sites and related sites and organs, components of personal, health and health-care data of individuals and diagnoses of diseases and illnesses in computer databases wherein said sites, symptoms and signs are selected, described, represented, expressed or chosen by said individuals for generating and displaying lists of diagnoses adaptable to be displayed on a means for displaying said sites, symptoms, signs, data and lists by a means for displaying said sites, symptoms, signs, data and lists.

### DESCRIPTION OF THE PREFERRED EMBODIMENTS

The present invention is a system and a method of generating and displaying lists of diagnoses and differential diagnoses (plural of diagnosis) of diseases, disorders and ailments relating to or affecting anatomical sites, body parts and organs relating to said diagnoses and differential diagnoses adaptable to be viewed by individuals.

Said anatomical sites include anatomical regions, landmarks and parts of human anatomy or body and organs on human figures, figurines, images, body parts, photographs, illustrations, displays, descriptions, representations, and words and/or terms describing or representing said anatomical sites adaptable to be displayed on a means for displaying said sites, figures, etc.

Furthermore, the present invention also provides the links of the symptoms, signs, manifestations and syndromes relating to diseases, disorders and illnesses relating to or affecting said site or sites wherein said site or sites, symptoms, signs, manifestations and syndromes are viewed selected, described, represented, expressed or any combination thereof by individuals.

Yet furthermore, the present invention also provides the links of elements or components in personal and health and health-care data of said individuals or patients in computer or server databases to achieve the goals and objectives of the present invention. Said personal data of an individual or patient is such as, but not limited to, date of birth or age, gender, social security number, unique identification number or numbers generated by said individual, place of birth, weights, heights and place of birth.

Said health and health-care data and information of individuals are generated by patients and physicians as taught in the earlier pending patent applications of the present applicants. Said data and information are such as, but not limited to, all medical and surgical histories, diagnoses, diseases, disorders, ailments, allergies, medications, treatments, laboratory, test and procedure data, results, interpretations, impressions and assessments such as, but not limited to, data, results, interpretations, impressions and assessments produce by blood tests, CTScan, MRI, endoscopies, arthroscopies, electrodiagnosis which are present in the databases on said individuals or patients.

The generations, storages, retrievals, editing, managements and viewing of said digital personal, health and health-care information and data by patients and physicians are taught by pending patent applications of the present inventors et al. Said medicine databases comprise encyclopedias, data and information use and practice by hundreds of millions of physicians and surgeons from about 80 medical and surgical specialties, fields or practices including physicians' and surgeons' clinical experiences and repertoires of knowledge and wisdom. Said databases are stored, linked to said sites, data and information, etc., queried and retrieved to generate and display said lists of diagnoses and differential diagnoses (plural of diagnosis) of diseases, disorders and ailments relating to or affecting anatomical sites, body parts and organs relating to said diagnoses and differential diagnoses.

The present invention, using various commercially available computer software and programs and methods, applies said anatomical site or sites, symptoms, signs, syndromes and manifestations relating to said site or sites and elements and components of personal, health and health-care data of said individuals and said medicine databases generated, linked, stored and retrievable from the databases of the computers or servers in a network of computers and servers or networks of computers and servers to achieve the goals and objectives of the present invention.

Said information and data are viewed on a means for displaying said sites, symptoms, signs, data, information and lists such as, but not limited to, the screens of computers, servers, laptops and the like and various other electronic mobile equipment or devices such as, but not limited to, cellular phones, mobile phones, smart phones, iPod (Apple Computer Corp.), Zune (Microsoft Corp.) and the like.

Although the preferred embodiments of this invention have been described to be used and applied to humans, it will be appreciated by those skilled in the art and science that the present invention including its adaptations and variations may be made to be used and applied to animals without departing from the spirit and the scope of the claims of the present invention.

Although the preferred embodiments of this invention have been described, it will be appreciated by those skilled in the art and science that adaptations and variations may be made without departing from the spirit and the scope of the claims of the present invention.

1. A method of generating lists of diagnoses relating to anatomical sites and symptoms and signs relating to diseases and disorders relating to said sites wherein said sites, symptoms and signs are selected, described, represented or expressed by individuals adaptable to be displayed on a means for displaying said sites, symptoms, signs, data and lists by a means for displaying said sites, symptoms, signs, data and lists comprises:
a process of selecting an anatomical site on a human image;
a process of selecting anatomical sites on a human image;
a process of selecting an anatomical site on a plurality of human images;
a process of selecting anatomical sites on a plurality of human images;
a process of showing a list of symptoms and signs relating to an anatomical site;
a process of showing lists of symptoms and signs relating to an anatomical site;
a process of showing lists of symptoms and signs relating to or affecting anatomical sites;
a process of relating an anatomical site and symptoms and signs relating to said site to diseases and disorders relating to or affecting said site;
a process of relating anatomical sites and symptoms and signs relating to said sites to diseases and disorders relating to or affecting said sites;
a process of using personal and health and health-care data of an individual;
a process of using personal and health and health-care data of individuals;
a process of linking personal and health and health-care data of an individual to an anatomical site and symptoms and signs relating to said site to diseases and disorders relating to or affecting said site;
a process of linking personal and health and health-care data of an individual to anatomical sites and symptoms and signs relating to said sites and to diseases and disorders relating to or affecting said sites;
a process of using personal and health and health-care data of individuals; and
a process of linking personal and health and health-care data of individuals to anatomical sites and symptoms and signs relating to said sites and to diseases and disorders relating to or affecting said sites.

2. A method of generating lists of diagnoses relating to anatomical sites and symptoms and signs relating to diseases and disorders relating to said sites and to personal and health and health-care data of individuals wherein said sites, symptoms and signs are selected, described, represented or expressed by said individuals and wherein said sites, symptoms, signs, data and lists are displayed on a means for displaying said sites, symptoms, signs, data and lists by a means for displaying said sites, symptoms, signs, data and lists comprises:
a process of selecting an anatomical site on a human image;
a process of selecting anatomical sites on a human image;
a process of selecting an anatomical site on a plurality of human images;
a process of selecting anatomical sites on a plurality of human images;
a process of showing a list of symptoms and signs relating to an anatomical site;
a process of showing lists of symptoms and signs relating to an anatomical site;
a process of showing lists of symptoms and signs relating to or affecting anatomical sites;
a process of relating an anatomical site and symptoms and signs relating to said site to diseases and disorders relating to or affecting said site;
a process of relating anatomical sites and symptoms and signs relating to said sites to diseases and disorders relating to or affecting said sites;
a process of using personal and health and health-care data of an individual;
a process of using personal and health and health-care data of individuals;
a process of linking personal and health and health-care data of an individual to an anatomical site and symptoms and signs relating to said site to diseases and disorders relating to or affecting said site;
a process of linking personal and health and health-care data of an individual to anatomical sites and symptoms and signs relating to said sites and to diseases and disorders relating to or affecting said sites;
a process of using personal and health and health-care data of individuals; and
a process of linking personal and health and health-care data of individuals to anatomical sites and symptoms and signs relating to said sites and to diseases and disorders relating to or affecting said sites.

3. A method of linking anatomical sites on human images, symptoms and signs relating to said sites and personal, health and health-care data of individuals and diagnoses of diseases and illnesses relating to said sites, symptoms, signs and components of said data wherein said sites, symptoms and signs are selected, described, represented, expressed or chose by said individuals for generating and displaying lists of diagnoses adaptable to be displayed on a means for displaying said sites, symptoms, signs, data and lists by a means for displaying said sites, symptoms, signs, data and lists comprises:
a process of selecting an anatomical site on a human image;
a process of selecting anatomical sites on a human image;
a process of selecting an anatomical site on a plurality of human images;
a process of selecting anatomical sites on a plurality of human images;
a process of showing a list of symptoms and signs relating to an anatomical site;
a process of showing lists of symptoms and signs relating to an anatomical site;
a process of showing lists of symptoms and signs relating to or affecting anatomical sites;
a process of relating an anatomical site and symptoms and signs relating to said site to diseases and disorders relating to or affecting said site;
a process of relating anatomical sites and symptoms and signs relating to said sites to diseases and disorders relating to or affecting said sites;
a process of using personal and health and health-care data of an individual;
a process of using personal and health and health-care data of individuals;
a process of linking personal and health and health-care data of an individual to an anatomical site and symptoms and signs relating to said site to diseases and disorders relating to or affecting said site;
a process of linking personal and health and health-care data of an individual to anatomical sites and symptoms and signs relating to said sites and to diseases and disorders relating to or affecting said sites;
a process of using personal and health and health-care data of individuals; and
a process of linking personal and health and health-care data of individuals to anatomical sites and symptoms and signs relating to said sites and to diseases and disorders relating to or affecting said sites.
